(19) United States
(12) Patent Application Publication
(10) Pub. No.: US 2017/0042915 A1
(43) Pub. Date: Feb. 16, 2017

(54) LPA RECEPTOR MODULATORS FOR BROWN FAT DIFFERENTIATION

(71) Applicant: The California Institute for Biomedical Research, La Jolla, CA (US)

(72) Inventors: Matthew S. TREMBLAY, San Diego, CA (US); Nicole ALVAREZ, San Marcos, CA (US); Weijun SHEN, San Diego, CA (US); Andrew TO, San Diego, CA (US)

(21) Appl. No.: 15/234,994

(22) Filed: Aug. 11, 2016

Related U.S. Application Data
(60) Provisional application No. 62/204,120, filed on Aug. 12, 2015.

(51) Int. Cl. A61K 31/683; A61K 31/42; A61K 31/415; A61K 31/473; A61K 31/4035; A61K 31/196; A61K 31/454; A61K 31/427

(52) U.S. Cl. CPC A61K 31/683; A61K 31/454; A61K 31/42; A61K 31/427; A61K 31/473; A61K 31/4035; A61K 31/196; A61K 31/415

(57) ABSTRACT

Described herein is a method of increasing brown fat differentiation or increasing energy expenditure in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof.

### CROSS-REFERENCE

This application claims benefit of U.S. Provisional Application No. 62/204,120, filed on Aug. 12, 2015, which is herein incorporated by reference in its entirety.

### SEQUENCE LISTING

The instant application contains a Sequence Listing which has been submitted electronically in ASCII format and is hereby incorporated by reference in its entirety. Said ASCII copy, created on Sep. 21, 2016, is named 41135-752_201_SL.txt and is 1,730 bytes in size.

### BACKGROUND OF THE INVENTION

Obesity remains a major health challenge in the western world. Obesity contributes to diseases such as coronary artery disease, hypertension, stroke, diabetes, hyperlipidemia and some cancers. The development of effective and safe therapies for obesity may be useful as first-line agents or in combination with other treatments.

### SUMMARY OF THE INVENTION

Provided herein is a method of increasing brown fat differentiation in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof.

In some embodiments is a method of increasing brown fat differentiation in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator has the structure:

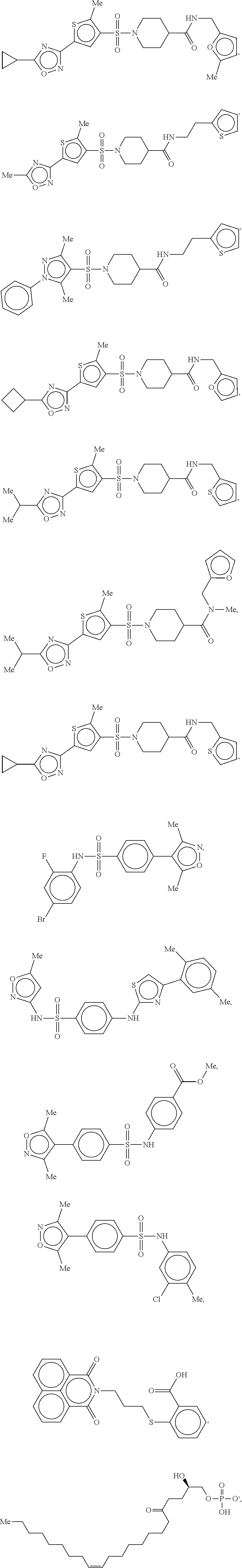

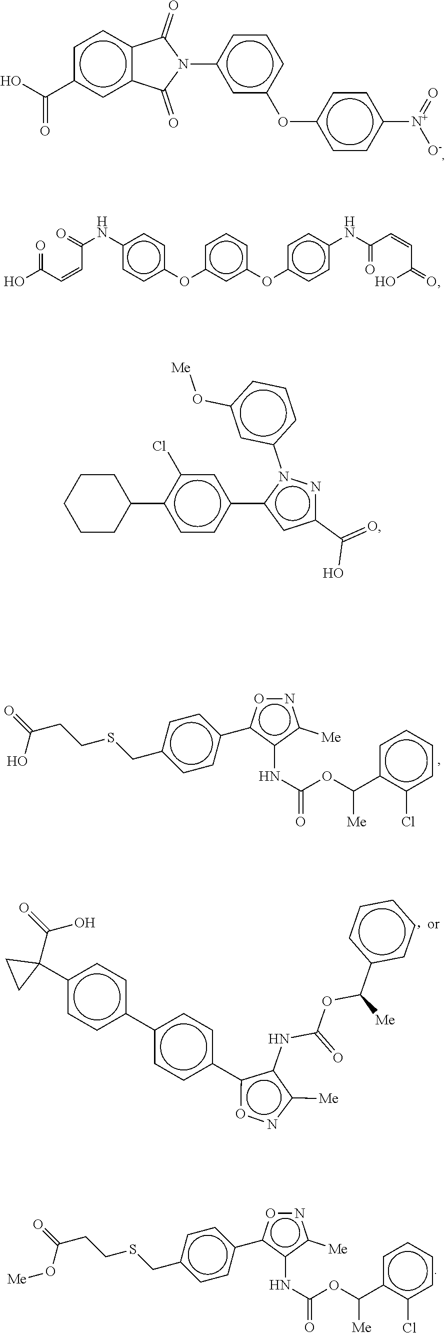

In some embodiments is a method of increasing brown fat differentiation in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator has the structure:

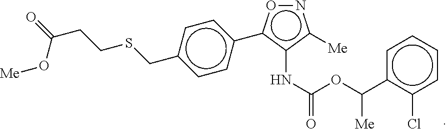

In some embodiments is a method of increasing brown fat differentiation in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator has the structure:

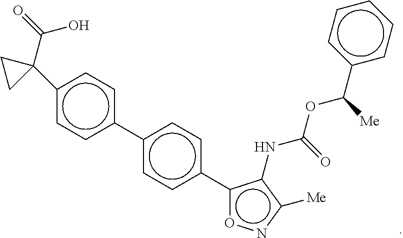

In some embodiments is a method of increasing brown fat differentiation in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator has the structure:

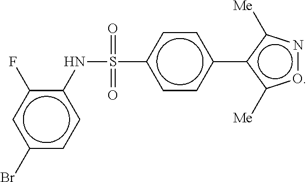

In some embodiments is a method of increasing brown fat differentiation in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator promotes or increases weight loss in the subject. In some embodiments is a method of increasing brown fat differentiation in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator promotes or increases insulin sensitization. In some embodiments is a method of increasing brown fat differentiation in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator is minimally systemically absorbed. In some embodiments is a method of increasing brown fat differentiation in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator is an LPA receptor antagonist.

In some embodiments provided herein is a method for increasing energy expenditure in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, to activate differentiation of the white adipose tissue into brown fat cells in the subject, wherein the differentiated brown fat cells promote energy expenditure thereby increasing energy expenditure in the mammal.

In some embodiments is a method for increasing energy expenditure in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator has the structure:

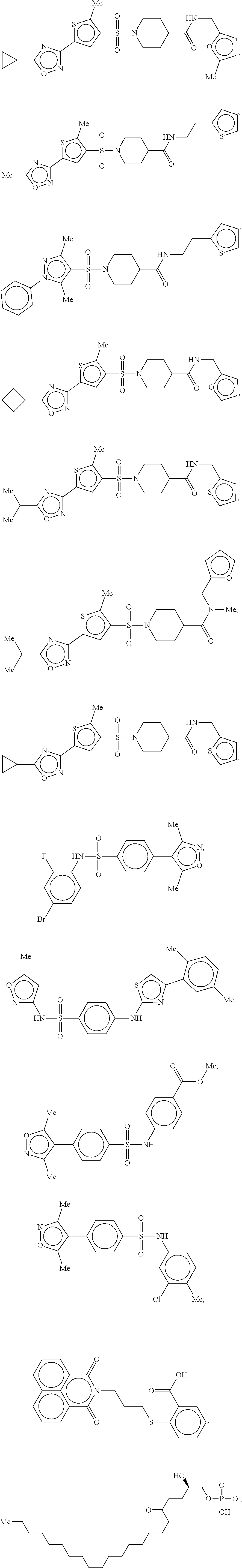

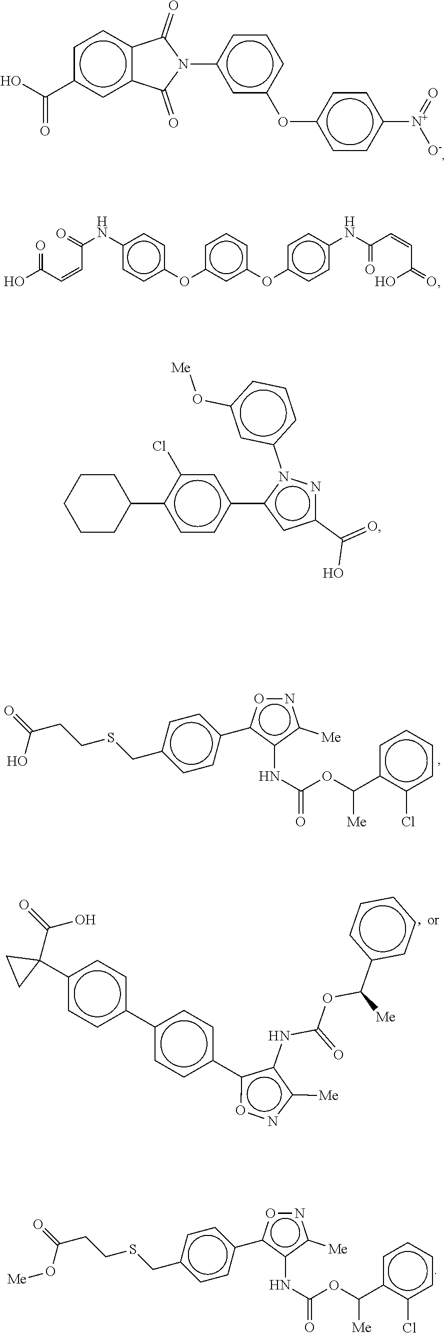

In some embodiments is a method for increasing energy expenditure in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator has the structure:

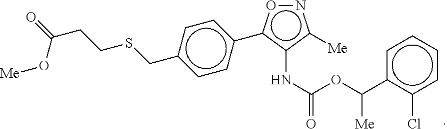

In some embodiments is a method for increasing energy expenditure in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator has the structure:

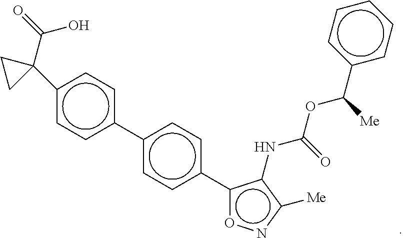

In some embodiments is a method for increasing energy expenditure in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator has the structure:

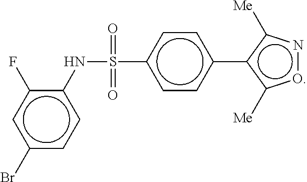

In some embodiments is a method for increasing energy expenditure in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator promotes or increases weight loss in the subject. In some embodiments is a method for increasing energy expenditure in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator promotes or increases insulin sensitization. In some embodiments is a method for increasing energy expenditure in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator is minimally systemically absorbed. In some embodiments is a method for increasing energy expenditure in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator is an LPA receptor antagonist.

Also provided herein in some embodiments is an LPA receptor modulator conjugated to a lipophilic derivative or substituent, or a pharmaceutically acceptable salt, solvate, polymorph, prodrug, metabolite, deuteride, N-oxide, stereoisomer, or isomer thereof. In some embodiments, the LPA receptor modulator is an LPA receptor antagonist. In other embodiments, the LPA receptor modulator is an LPA receptor agonist.

In some embodiments, the LPA receptor modulator has the structure of:

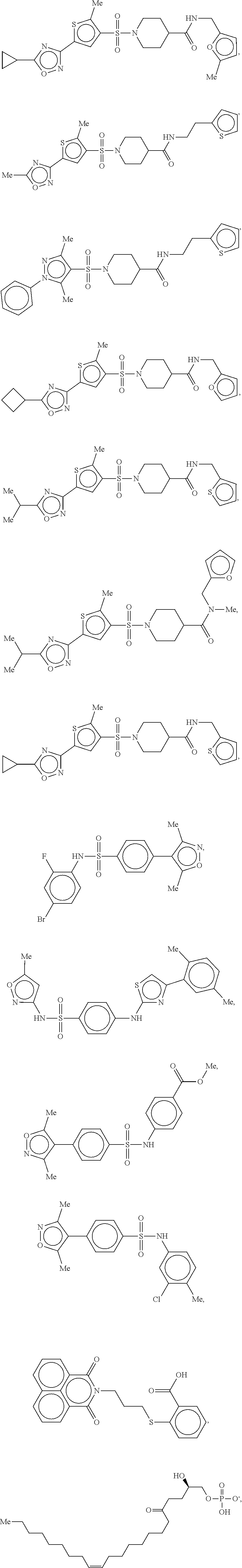

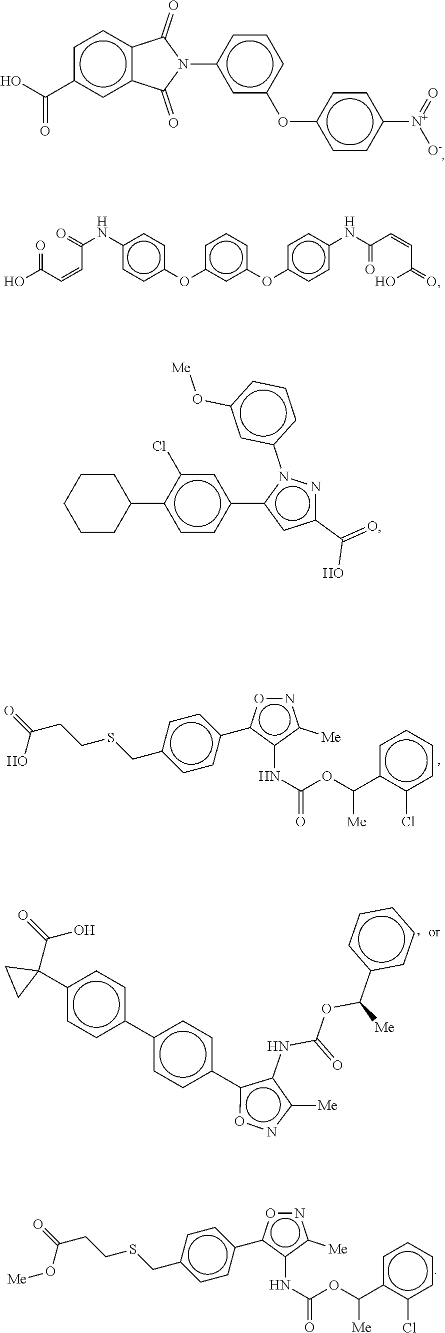

In some embodiments, the LPA receptor modulator or a pharmaceutically acceptable salt, solvate, polymorph, prodrug, metabolite, deuteride, N-oxide, stereoisomer, or isomer thereof, wherein the modulator has the structure of:

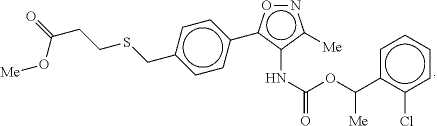

In other embodiments, the LPA receptor modulator or a pharmaceutically acceptable salt, solvate, polymorph, prodrug, metabolite, deuteride, N-oxide, stereoisomer, or isomer thereof, wherein the modulator has the structure of:

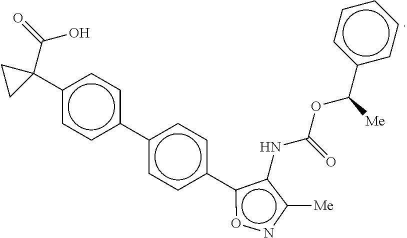

In some embodiments, the LPA receptor modulator or a pharmaceutically acceptable salt, solvate, polymorph, prodrug, metabolite, deuteride, N-oxide, stereoisomer, or isomer thereof, wherein the modulator has the structure of:

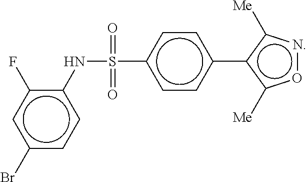

Some embodiments provided herein describe an LPA receptor modulator (e.g., LPA receptor antagonist) conjugated to a lipid. In some embodiments, the LPA receptor modulator (e.g., LPA receptor antagonist) is conjugated to a fatty acid derivative. In certain embodiments, the LPA receptor modulator (e.g., LPA receptor antagonist) is conjugated to a fatty acid.

Also described herein in some embodiments are pharmaceutical compositions comprising an LPA receptor modulator (e.g., LPA receptor antagonist) and a pharmaceutically acceptable excipient.

Also described herein in some embodiments are methods of increasing brown fat differentiation in a subject in need thereof, the method comprising administering to the subject a composition comprising an LPA receptor modulator conjugated to a lipophilic derivative or substituent. In some embodiments, the LPA receptor modulator is an antagonist. In other embodiments, the LPA receptor modulator is an agonist. In some instances, the subject is monitored for differentiation of brown fat cells. In some instances, the LPA receptor modulator conjugated to a lipophilic derivative or substituent is injected into the subject subcutaneously. In some instances, the LPA receptor modulator is administered to the subject orally.

In some embodiments, the LPA receptor modulator is an antagonist. In other embodiments, the LPA receptor modulator is an agonist.

Other embodiments provided herein describe methods of treating obesity or an obesity related disease or disorder in a subject, the method comprising administering to the subject an LPA receptor modulator conjugated to a lipophilic derivative or substituent. In certain embodiments, the LPA receptor modulator increases respiration and energy expenditure to thereby treat obesity or the obesity-related disorder. Also described herein in some embodiments are methods of treating type II diabetes in a subject, the method comprising administering to the subject an LPA receptor modulator conjugated to a lipophilic derivative or substituent. Also described herein in some embodiments are methods of treating protein energy malnutrition in a subject, the method comprising administering to the subject an LPA receptor modulator conjugated to a lipophilic derivative or substituent. In some embodiments, the LPA receptor modulator is an antagonist. In other embodiments, the LPA receptor modulator is an agonist.

In certain embodiments, the LPA receptor modulator promotes or increases weight loss in the subject. In some embodiments, the LPA receptor modulator promotes or increases insulin sensitization. In some embodiments, the LPA receptor modulator is minimally systemically absorbed.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates the total percentage of adipocytes, the percentage of white adipocytes, and the percentage of brown adipocytes observed when human Simpson-Golabi-Behmel syndrome (SGBS) preadipocytes were caused to undergo adipogenesis via standard methods in the presence or absence of Compound 19 wherein the quantified percentages are based on anti-UCP1 and LipidTox staining.

FIG. 1B illustrates the total percentage of adipocytes, the percentage of white adipocytes, and the percentage of brown adipocytes observed when human SGBS preadipocytes were caused to undergo adipogenesis via standard methods in the presence or absence of Compound 19 wherein the quantified percentages are based on MitoTracker and LipidTox staining.

FIG. 2 shows mRNA expression of markers for brown adipocytes (UCP1) and adipogenesis (FABP4, PPARγ) in SGBS preadipocytes (day 0) versus day 11 adipocytes differentiated in the presence of Rosi (positive control) or Compound 19.

FIG. 3 shows UCP1 protein expression in SGBS preadipocytes (day 0) versus day 11 adipocytes differentiated in the presence of Rosi (positive control) or Compound 19.

FIG. 4 illustrates percentages of brown, white, and total adipocytes in human primary preadipocytes differentiated in the presence of various concentrations of Compound 19. Percentages are based on Lipidtox and UCP1 parameters on day 14 post-differentiation.

FIG. 5A illustrates cellular respiration rates in SGBS adipocytes differentiated in the presence of Compound 19 or vehicle (DMSO) (day 11 post-differentiation). Oxygen consumption rates (OCR) were measured using the Seahorse flux analyzer XF96 with sequential injections of 2 μM oligomycin, 1 μM FCCP, and 0.5 μM RAA as indicated.

FIG. 5B is the same as in FIG. 5a but adipocytes were pre-treated with forskolin (10 μM) for 1 hour prior to measuring OCR as shown.

FIG. 5C shows the calculated percent of total respiration that is uncoupled for data shown in FIG. 5a-b.

FIG. 6A illustrates attenuated induction of brown adipocyte differentiation by Compound 19 in the presence of exogenous LPA. Percent brown adipocytes in day 11 SGBS adipocytes differentiated in the presence of Compound 19 during days 3-4 of induction with (right) or without (left) addition of LPA to the medium during days 1-4 of induction.

FIG. 6B illustrates recapitulation of the browning phenotype using a combination of ATX and PLA inhibitors to reduce endogenous LPA production. Percent brown adipocytes shown for day 11 SGBS adipocytes differentiated in the presence of rosiglitazone (2 μM), Compound 19 (10 μM), ATX inhibitor (10 μM), PLA inhibitor (10 μM), or both ATX and PLA inhibitors.

### DETAILED DESCRIPTION OF THE INVENTION

Generally, obesity results when energy intake exceeds energy expenditure, resulting in the growth and/or formation of adipose tissue via hypertrophic and hyperplastic growth. Hypertrophic growth is an increase in size of adipocytes stimulated by lipid accumulation. Hyperplastic growth is defined as an increase in the number of adipocytes in adipose tissue.

Adipose (fat) tissue consists primarily of adipocytes. Vertebrates possess two distinct types of adipose tissue: white adipose tissue (WAT) and brown adipose tissue (BAT). WAT stores and releases fat according to the nutritional needs of the animal. This stored fat is used by the body for (1) heat insulation (e.g., subcutaneous fat), (2) mechanical cushion (e.g., surrounding internal organs), and (3) as a source of energy. BAT burns fat, releasing the energy as heat through thermogenesis. BAT thermogenesis is used both (1) to maintain homeothermy by increasing thermogenesis in response to lower temperatures and (2) to maintain energy balance by increasing energy expenditure in response to increases in caloric intake. BAT is also the major site of thermogenesis in rodents and plays an important role in thermogenesis in human infants. In humans, brown fat diminishes with age.

Fat metabolism is regulated by two pathways, lipogenesis and lipolysis. Lipogenesis is the deposition of fat which occurs in the liver and in adipose tissue at cytoplasmic and mitochondrial sites. This process allows the storage of energy that is ingested which is not needed for current energy demands. Lipolysis is the chemical decomposition and release of fat from adipose and/or other tissues. This process predominates over lipogenesis when additional energy is required by the body.

In some instances, increased brown fat differentiation induces the expression of mitochondrial genes and cellular respiration in mammals. Densely packed mitochondria are a characteristic of brown fat cells. Increased respiration results in increased heat dissipation and increased energy expenditure by the mammal. The increases in heat dissipation and increased energy expenditure stimulate the metabolic rate of the mammal. In some instances, agents that increase brown fat differentiation may be used to treat and/or prevent obesity or an obesity related disorder through the stimulation of the metabolic rate.

Described herein are certain compounds, compositions, and methods for treating and/or preventing obesity or obesity related disorders by increasing energy expenditure or metabolic activity. In some embodiments, the compounds and compositions described herein increase brown fat differentiation in mammals. In some embodiments, the compounds and compositions described herein stimulate the metabolic rate. In some embodiments, the compounds described herein are modulators of (lysophosphatidic acid) LPA receptors. In some embodiments, the compounds are LPA receptor modulators (e.g., antagonists) and are useful anti-obesity therapeutics. In some embodiments, the compounds and compositions described herein comprise a lipid or fatty acid conjugate of an LPA receptor modulator (e.g., LPA receptor antagonist).

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a”, “an”, and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“Amino” refers to the —NH₂ radical.

“Cyano” or “nitrile” refers to the —CN radical.

“Hydroxy” or “hydroxyl” refers to the —OH radical.

“Nitro” refers to the —NO₂ radical.

“Oxo” refers to the ═O substituent.

“Oxime” refers to the ═N—OH substituent.

“Thioxo” refers to the ═S substituent.

“Alkyl” refers to a straight or branched hydrocarbon chain radical, has from one to thirty carbon atoms, and is attached to the rest of the molecule by a single bond. Alkyls comprising any number of carbon atoms from 1 to 30 are included. An alkyl comprising up to 30 carbon atoms is referred to as a C₁-C₃₀ alkyl, likewise, for example, an alkyl comprising up to 12 carbon atoms is a C₁-C₁₂ alkyl. Alkyls (and other moieties defined herein) comprising other numbers of carbon atoms are represented similarly. Alkyl groups include, but are not limited to, C₁-C₃₀ alkyl, C₁-C₂₀ alkyl, C₁-C₁₅ alkyl, C₁-C₁₀ alkyl, C₁-C₈ alkyl, C₁-C₆ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (iso-propyl), n-butyl, i-butyl, s-butyl, n-pentyl, 1,1-dimethylethyl (t-butyl), 3-methylhexyl, 2-methylhexyl, vinyl, allyl, propynyl, and the like. Alkyl comprising unsaturations include alkenyl and alkynyl groups. Unless stated otherwise specifically in the specification, an alkyl group may be optionally substituted as described below.

“Alkylene” or “alkylene chain” refers to a straight or branched divalent hydrocarbon chain, as described for alkyl above. Unless stated otherwise specifically in the specification, an alkylene group may be optionally substituted as described below.

“Alkoxy” refers to a radical of the formula —ORₐ where Rₐ is an alkyl radical as defined. Unless stated otherwise specifically in the specification, an alkoxy group may be optionally substituted as described below.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused or bridged ring systems. Aryl radicals include, but are not limited to, aryl radicals derived from the hydrocarbon ring systems of aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

“Cycloalkyl” or “carbocycle” refers to a stable, non-aromatic, monocyclic or polycyclic carbocyclic ring, which may include fused or bridged ring systems, which is saturated or unsaturated. Representative cycloalkyls or carbocycles include, but are not limited to, cycloalkyls having from three to fifteen carbon atoms, from three to ten carbon atoms, from three to eight carbon atoms, from three to six carbon atoms, from three to five carbon atoms, or three to four carbon atoms. Monocyclic cycloalkyls or carbocycles include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Unless otherwise stated specifically in the specification, a cycloalkyl or carbocycle group may be optionally substituted. Illustrative examples of cycloalkyl groups include, but are not limited to, the following moieties:

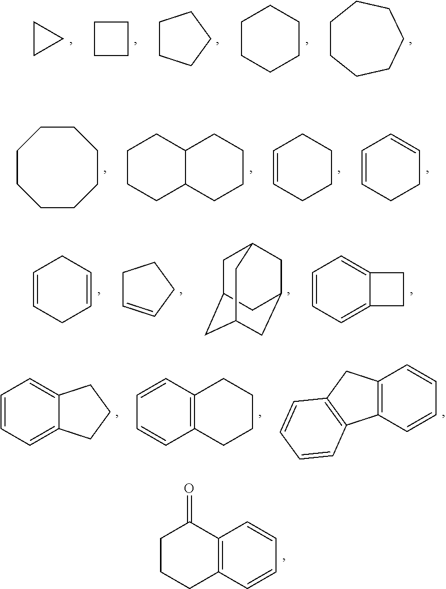

and the like.

“Fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” similarly refers to a radical of the formula —ORₐ where Rₐ is a haloalkyl radical as defined. Unless stated otherwise specifically in the specification, a haloalkoxy group may be optionally substituted as described below.

“Heterocycloalkyl” or “heterocyclyl” or “heterocyclic ring” or “heterocycle” refers to a stable 3- to 24-membered non-aromatic ring radical comprising 2 to 23 carbon atoms and from one to 8 heteroatoms selected from the group consisting of nitrogen, oxygen, phosphorous and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 12-crown-4, 15-crown-5, 18-crown-6, 21-crown-7, aza-18-crown-6, diaza-18-crown-6, aza-21-crown-7, and diaza-21-crown-7. Unless stated otherwise specifically in the specification, a heterocyclyl group may be optionally substituted. Illustrative examples of heterocycloalkyl groups, also referred to as non-aromatic heterocycles, include:

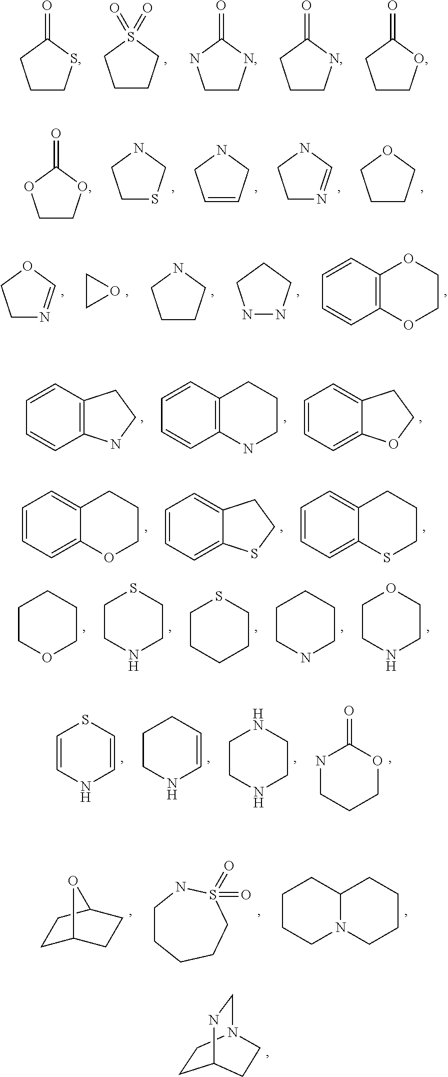

and the like. The term heterocycloalkyl also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “heteroaryl” as used herein, alone or in combination, refers to optionally substituted aromatic monoradicals containing from about five to about twenty skeletal ring atoms, where one or more of the ring atoms is a heteroatom independently selected from among oxygen, nitrogen, sulfur, phosphorous, silicon, selenium and tin but not limited to these atoms and with the proviso that the ring of said group does not contain two adjacent O or S atoms. In embodiments in which two or more heteroatoms are present in the ring, the two or more heteroatoms can be the same as each another, or some or all of the two or more heteroatoms can each be different from the others. The term heteroaryl includes optionally substituted fused and non-fused heteroaryl radicals having at least one heteroatom. The term heteroaryl also includes fused and non-fused heteroaryls having from five to about twelve skeletal ring atoms, as well as those having from five to about ten skeletal ring atoms. Bonding to a heteroaryl group can be via a carbon atom or a heteroatom. Thus, as a non-limiting example, an imidazole group may be attached to a parent molecule via any of its carbon atoms (imidazol-2-yl, imidazol-4-yl or imidazol-5-yl), or its nitrogen atoms (imidazol-1-yl or imidazol-3-yl). Likewise, a heteroaryl group may be further substituted via any or all of its carbon atoms, and/or any or all of its heteroatoms. A fused heteroaryl radical may contain from two to four fused rings where the ring of attachment is a heteroaromatic ring and the other individual rings may be alicyclic, heterocyclic, aromatic, heteroaromatic or any combination thereof. A non-limiting example of a single ring heteroaryl group includes pyridyl; fused ring heteroaryl groups include benzimidazolyl, quinolinyl, acridinyl; and a non-fused bi-heteroaryl group includes bipyridinyl. Further examples of heteroaryls include, without limitation, furanyl, thienyl, oxazolyl, acridinyl, azepinyl, phenazinyl, benzimidazolyl, benzindolyl,benzofuranyl, benzofuranonyl,benzoxazolyl, benzothiazolyl, benzothiadiazolyl, benzothiophenyl, benzoxadiazolyl, benzodioxolyl, benzo[b][1,4]dioxepinyl, 1,4-benzodioxanyl, benzonaphthofuranyl, benzotriazolyl, benzodioxolyl, benzodioxinyl, benzopyranyl, benzopyranonyl, benzothienyl (benzothiophenyl), benzo[4,6]imidazo[1,2-a]pyridinyl, carbazolyl, cinnolinyl, dibenzofuranyl, dibenzothiophenyl, furanonyl, imidazolyl, indolyl, isoxazolyl, isoquinolinyl, indolizinyl, indazolyl, isoindolyl, indolinyl, isoindolinyl, indolizinyl, isothiazolyl, isoindolyloxadiazolyl, indazolyl, naphthyridinyl, oxadiazolyl, 2-oxoazepinyl, oxiranyl, 1-oxidopyridinyl, 1-oxidopyrimidinyl, 1-oxidopyrazinyl, 1-oxidopyridazinyl, 1-phenyl-1H-pyrrolyl, phenothiazinyl, phenoxazinyl, pyridinyl, pyridazinyl, pyrimidinyl, pyrazinyl, pyrrolyl, pyrazinyl, pyrazolyl, purinyl, phthalazinyl, pteridinyl, quinolinyl, quinazolinyl, quinoxalinyl, quinuclidinyl,triazolyl, tetrazolyl, thiazolyl, triazinyl, thiadiazolyl, tetrahydroquinolinyl, thiazolyl, and thiophenyl and the like, and their oxides, such as for example pyridyl-N-oxide. Illustrative examples of heteroaryl groups include the following moieties:

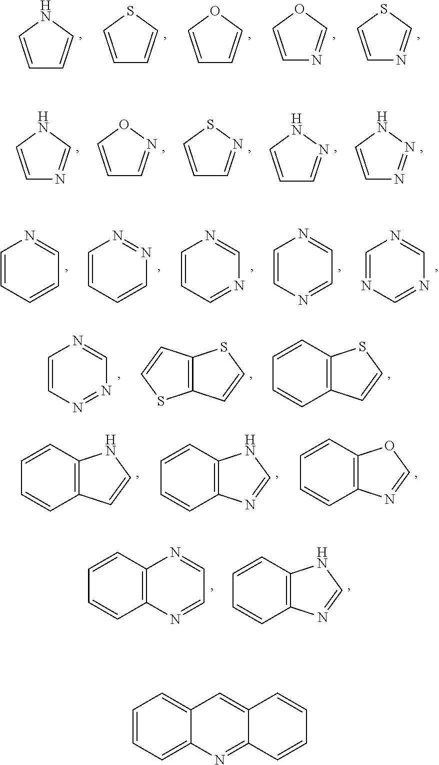

and the like.

All the above groups may be either substituted or unsubstituted. The term “substituted” as used herein means any of the above groups (e.g, alkyl, alkylene, alkoxy, aryl, cycloalkyl, haloalkyl, heterocyclyl and/or heteroaryl) may be further functionalized wherein at least one hydrogen atom is replaced by a bond to a non-hydrogen atom substituent. Unless stated specifically in the specification, a substituted group may include one or more substituents selected from: oxo, amino, —CO₂H, nitrile, nitro, hydroxyl, thiooxy, alkyl, alkylene, alkoxy, aryl, cycloalkyl, heterocyclyl, heteroaryl, dialkylamines, arylamines, alkylarylamines, diarylamines, trialkylammonium (—N⁺R₃), N-oxides, imides, and enamines; a silicon atom in groups such as trialkylsilyl groups, dialkylarylsilyl groups, alkyldiarylsilyl groups, triarylsilyl groups, perfluoroalkyl or perfluoroalkoxy, for example, trifluoromethyl or trifluoromethoxy. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NH₂, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —S O₂R_{g}, —OSO₂R_{g}, —SO₂OR_{g}, ═NSO₂R_{g}, and —SO₂NR_{g}Rₕ. In the foregoing, R_{g} and Rₕ are the same or different and independently hydrogen, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. In addition, each of the foregoing substituents may also be optionally substituted with one or more of the above substituents. Furthermore, any of the above groups may be substituted to include one or more internal oxygen, sulfur, or nitrogen atoms. For example, an alkyl group may be substituted with one or more internal oxygen atoms to form an ether or polyether group. Similarly, an alkyl group may be substituted with one or more internal sulfur atoms to form a thioether, disulfide, etc.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

An “effective amount” or “therapeutically effective amount” refers to an amount of a compound administered to a mammalian subject, either as a single dose or as part of a series of doses, which is effective to produce a desired therapeutic effect.

The terms “metabolic disorder” and “obesity related disorders” are used interchangeably herein and include a disorder, disease or condition which is caused or characterized by an abnormal metabolism (i.e., the chemical changes in living cells by which energy is provided for vital processes and activities) in a subject. Metabolic disorders include diseases, disorders, or conditions associated with aberrant thermogenesis or aberrant adipose cell (e.g., brown or white adipose cell) content or function. Metabolic disorders can detrimentally affect cellular functions such as cellular proliferation, growth, differentiation, or migration, cellular regulation of homeostasis, inter- or intra-cellular communication; tissue function, such as liver function, muscle function, renal function, or adipocyte function; systemic responses in an organism, such as hormonal responses (e.g., insulin response). Examples of metabolic disorders include obesity, including insulin resistant obesity, type I diabetes, type II diabetes, protein energy malnutrition, hyperphagia, coronary heart diseases, high blood pressure, gallstones, endocrine abnormalities, triglyceride storage disease, Bardet-Biedl syndrome, Lawrence-Moon syndrome, Prader-Labhart-Willi syndrome, anorexia, cachexia, and certain cancers.

As used herein, “obesity” refers to body weight that is greater than what is considered healthy for a certain height (i.e., a body mass index (BMI) of 30 kg/²m or more (National Institute of Health, Clinical Guidelines on the Identification, Evaluation, and Treatment of Overweight and Obesity in Adults (1998))). However, the present invention is also intended to include a disease, disorder, or condition that is characterized by a body mass index (BMI) of 25 kg/²m or more, 26 kg/²m or more, 27 kg/²m or more, 28 kg/²m or more, 29 kg/²m or more, 29.5 kg/²m or more, or 29.9 kg/²m or more, all of which are typically referred to as overweight (National Institute of Health, Clinical Guidelines on the Identification, Evaluation, and Treatment of Overweight and Obesity in Adults (1998)). The obesity described herein may be due to any cause, whether genetic or environmental. Examples of disorders that may result in obesity or be the cause of obesity include overeating and bulimia, polycystic ovarian disease, craniopharyngioma, the Prader-Willi Syndrome, Frohlich's syndrome, Type II diabetics, GH-deficient subjects, normal variant short stature, Turner's syndrome, and other pathological conditions showing reduced metabolic activity or a decrease in resting energy expenditure as a percentage of total fat-free mass, e.g., children with acute lymphoblastic leukemia.

As used herein, the terms “treat,” “treatment,” “treating,” or “amelioration” when used in reference to a disease, disorder or medical condition, refer to therapeutic treatments for a condition where the subject is in need of more brown adipocytes, wherein the object is to reverse, alleviate, ameliorate, inhibit, slow down or stop the progression or severity of a symptom or condition. The term “treating” includes reducing or alleviating at least one adverse effect or symptom of a condition. In the case of obesity or being overweight, the adverse effect includes not only clinical symptoms or markers of obesity-related disease, but also aesthetic indicators, such that a non-obese, but overweight individual's desire for weight loss or lower BMI is encompassed as a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), decrease in BMI, delay or slowing of the clinical progression of a condition, and amelioration or palliation of a condition. In some instances, the term “treatment” refers to reducing the BMI of the mammal to less than about 25.9, and maintaining that weight for a period of time, e.g., for at least about 6 months.

“Prevention” refers to preventing obesity or an obesity related disorder from occurring if the treatment is administered prior to the onset of the obese condition. Moreover, if treatment is commenced in subjects already suffering from or having symptoms of obesity or an obesity related disorder, such treatment is expected to prevent, or to prevent the progression of obesity or the obesity related disorder, and the medical sequelae of obesity, such as, e.g., arteriosclerosis, Type II diabetes, polycystic ovarian disease, cardiovascular diseases, osteoarthritis, dermatological disorders, hypertension, insulin resistance, hypercholesterolemia, hypertriglyceridemia, and cholelithiasis.

The terms “increased”, “increase” or “enhance” or “activate” or “promote” or “induce” are all used herein to generally mean an increase by a statically significant amount; for the avoidance of any doubt, the terms “increased”, “increase” or “enhance” or “activate” or “promote” or “induce” means an increase of at least 10% as compared to a reference level, for example an increase of at least about 20%, or at least about 30%, or at least about 40%, or at least about 50%, or at least about 60%, or at least about 70%, or at least about 80%, or at least about 90% or, up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of inducing or increasing differentiation, the reference level can be the extent of differentiation prior to treatment or in the absence of treatment according to the methods described herein.

A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

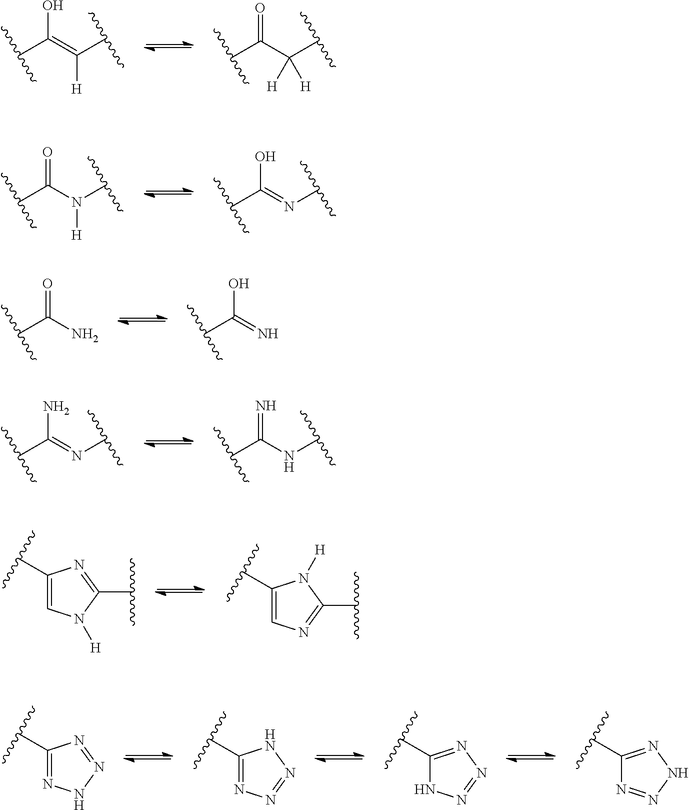

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes, such as, oxidation reactions) by which a particular substance is changed by an organism. Thus, enzymes may produce specific structural alterations to a compound. For example, cytochrome P450 catalyzes a variety of oxidative and reductive reactions while uridine diphosphate glucuronyl transferases catalyze the transfer of an activated glucuronic-acid molecule to aromatic alcohols, aliphatic alcohols, carboxylic acids, amines and free sulfhydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds. Both methods are well known in the art. In some embodiments, metabolites of a compound are formed by oxidative processes and correspond to the corresponding hydroxy-containing compound. In some embodiments, a compound is metabolized to pharmacologically active metabolites.

### Compounds

Described herein are anti-obesity therapeutics. In some embodiments, the anti-obesity therapeutic is any LPA receptor modulator. In some embodiments, the anti-obesity therapeutic is any LPA receptor antagonist. In some embodiments, the anti-obesity therapeutic is any LPA receptor agonist. Some embodiments provided herein describe methods of treating an obesity related disorder or disease in a subject, the method comprising administering to the subject an LPA receptor modulator (e.g., antagonist). In one embodiment, the LPA receptor modulator is conjugated to a lipophilic derivative or substituent. In one embodiment, the LPA receptor modulator is conjugated to a lipid or fatty acid derivative. In one embodiment, the LPA receptor modulator is conjugated to a lipid. In one embodiment, the LPA receptor modulator is conjugated to a fatty acid. In a further or additional embodiment, the LPA receptor modulator induces brown fat differentiation in the subject. In a further or additional embodiment, the LPA receptor modulator stimulates the subject's metabolic rate.

In one aspect, provided herein are LPA receptor modulators, or pharmaceutically acceptable salts, solvates, polymorphs, prodrugs, metabolites, N-oxides, stereoisomers, or isomers thereof, having the following structure:

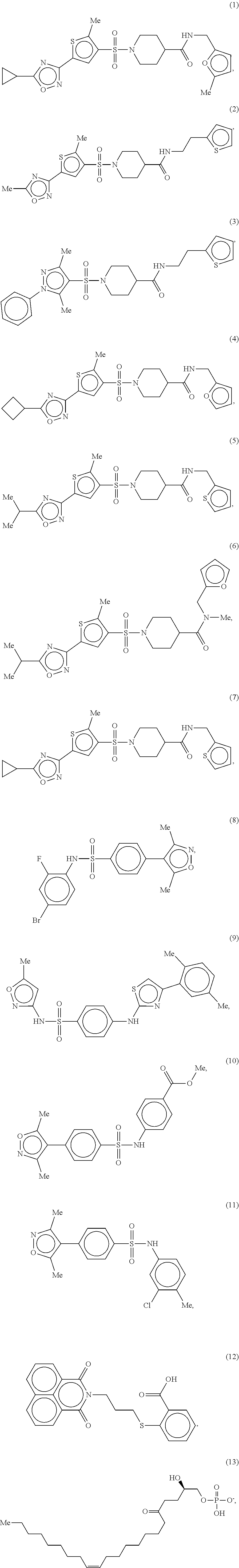

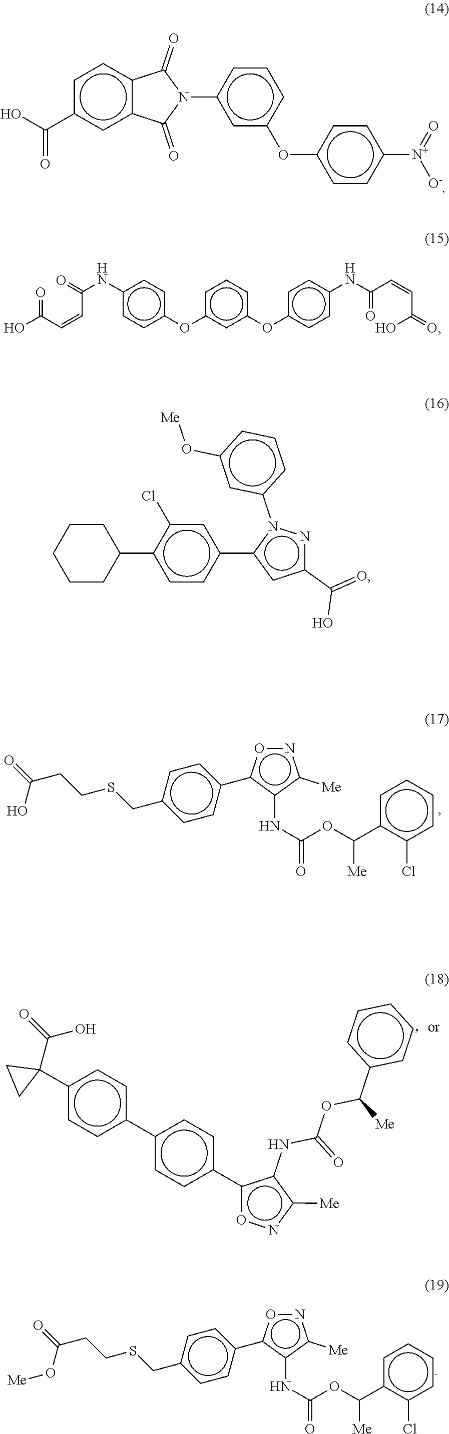

In some embodiments, the LPA receptor modulator is an LPA receptor antagonist. In other embodiments, the LPA receptor modulator is an LPA receptor agonist. In other embodiments, the LPA receptor modulator is a partial antagonist of the LPA receptor. In other embodiments, the LPA receptor modulator is a partial agonist of the LPA receptor.

In some embodiments, the compound is conjugated to a lipophilic substituent or derivative. In some embodiments, the compound is conjugated to a lipophilic derivative or substituent. In some embodiments, the compound is conjugated to a lipid. In some embodiments, the compound is conjugated to a fatty acid derivative. In a further or additional embodiment, the compound induces brown fat differentiation in the subject. In a further or additional embodiment, the compound stimulates the subject's metabolic rate.

In some embodiments, the compound does not exhibit PPARγ agonism.

### Preparation of Compounds

Pharmaceutical compositions comprising at least one LPA receptor antagonist or a pharmaceutically acceptable salt, pharmaceutically acceptable solvate, pharmaceutically active metabolite or pharmaceutically acceptable prodrug of such compound, and a pharmaceutically acceptable excipient are also provided.

In some instances, the LPA receptor antagonists described herein are synthesized using standard synthetic reactions known to those of skill in the art or using methods known in the art. In some instances, the reactions are employed in a linear sequence to provide the compounds. In other instances, the reactions are used to synthesize fragments which are subsequently joined by the methods known in the art.

The starting material used for the synthesis of the compounds described herein may be synthesized or obtained from commercial sources, such as, but not limited to, Aldrich Chemical Co. (Milwaukee, Wis.), Bachem (Torrance, Calif.), or Sigma Chemical Co. (St. Louis, Mo.). The compounds described herein, and other related compounds having different substituents can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001); Green and Wuts, PROTECTIVE GROUPS IN ORGANIC SYNTHESIS 3^{rd} Ed., (Wiley 1999); Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); and Larock's Comprehensive Organic Transformations (VCH Publishers Inc., 1989); (all of which are incorporated by reference in their entirety). Other methods for the synthesis of compounds described herein may be found in International Patent Publication No. WO 01/01982901, Arnold et al. Bioorganic & Medicinal Chemistry Letters 10 (2000) 2167-2170; Burchat et al. Bioorganic & Medicinal Chemistry Letters 12 (2002) 1687-1690. In some instances, general methods for the preparation of a compound as disclosed herein are derived from known reactions in the field, and the reactions may be modified by the use of appropriate reagents and conditions, as would be recognized by the skilled person, for the introduction of the various moieties found in the formulae as provided herein.

In some instances, the products of the reactions are isolated and purified, if desired, using conventional techniques, including, but not limited to, filtration, distillation, crystallization, chromatography and the like. In some instances, such materials are characterized using conventional means, including physical constants and spectral data.

In some instances, compounds described herein are prepared as a single isomer. In other instances, compounds described herein are prepared as a mixture of isomers.

The starting materials and intermediates for the compounds of this invention may be prepared by the application or adaptation of the methods described below, their obvious chemical equivalents, or, for example, as described in literature such as The Science of Synthesis, Volumes 1-8. Editors E. M. Carreira et al. Thieme publishers (2001-2008). Details of reagent and reaction options are also available by structure and reaction searches using commercial computer search engines such as SciFinder or Reaxys.

### Further Forms of Compounds Disclosed Herein

### Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In certain embodiments, the compounds described herein exist as partially or fully deuterated forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the metabolites, pharmaceutically acceptable salts, esters, prodrugs, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, prodrug, solvate, hydrate or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the invention, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, y-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate undeconate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds of the invention and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Polymorphs

In some embodiments, the compounds described herein exist as polymorphs. The invention provides for methods of treating diseases by administering such polymorphs. The invention further provides for methods of treating diseases by administering such polymorphs as pharmaceutical compositions.

Thus, the compounds described herein include all their crystalline forms, known as polymorphs. Polymorphs include the different crystal packing arrangements of the same elemental composition of a compound. In certain instances, polymorphs have different X-ray diffraction patterns, infrared spectra, melting points, density, hardness, crystal shape, optical and electrical properties, stability, and solubility. In certain instances, various factors such as the recrystallization solvent, rate of crystallization, and storage temperature cause a single crystal form to dominate.

### Prodrugs

In some embodiments, the compounds described herein exist in prodrug form. The invention provides for methods of treating diseases by administering such prodrugs. The invention further provides for methods of treating diseases by administering such prodrugs as pharmaceutical compositions.

Prodrugs are generally drug precursors that, following administration to an individual and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs are often useful because, in some situations, they are easier to administer than the parent drug. They are, for instance, bioavailable by oral administration whereas the parent is not. In certain instances, the prodrug also has improved solubility in pharmaceutical compositions over the parent drug. An example, without limitation, of a prodrug would be a compound as described herein which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyamino acid) bonded to an acid group where the peptide is metabolized to reveal the active moiety. (See for example Bundgaard, “Design and Application of Prodrugs” in A Textbook of Drug Design and Development, Krosgaard-Larsen and Bundgaard, Ed., 1991, Chapter 5, 113-191, which is incorporated herein by reference).

In some embodiments, prodrugs are designed as reversible drug derivatives, for use as modifiers to enhance drug transport to site-specific tissues. The design of prodrugs to date has been to increase the effective water solubility of the therapeutic compound for targeting to regions where water is the principal solvent.

Additionally, prodrug derivatives of compounds described herein can be prepared by methods described herein are otherwise known in the art (for further details see Saulnier et al., Bioorganic and Medicinal Chemistry Letters, 1994, 4, 1985). By way of example only, appropriate prodrugs can be prepared by reacting a non-derivatized compound with a suitable carbamylating agent, such as, but not limited to, 1,1-acyloxyalkylcarbanochloridate,para-nitrophenyl carbonate, or the like. Prodrug forms of the herein described compounds, wherein the prodrug is metabolized in vivo to produce a derivative as set forth herein are included within the scope of the claims. Indeed, some of the herein-described compounds are prodrugs for another derivative or active compound.

In some embodiments, prodrugs include compounds wherein an amino acid residue, or a polypeptide chain of two or more (e. g., two, three or four) amino acid residues is covalently joined through an amide or ester bond to a free amino, hydroxy or carboxylic acid group of compounds of the present invention. The amino acid residues include but are not limited to the 20 naturally occurring amino acids and also includes 4-hydroxyproline, hydroxylysine, demosine, isodemosine, 3-methylhistidine, norvaline, beta-alanine, gamma-aminobutyric acid, cirtulline, homocysteine, homoserine, ornithine and methionine sulfone. In other embodiments, prodrugs include compounds wherein a nucleic acid residue, or an oligonucleotide of two or more (e. g., two, three or four) nucleic acid residues is covalently joined to a compound of the present invention.

Pharmaceutically acceptable prodrugs of the compounds described herein also include, but are not limited to, esters, carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts and sulfonate esters. Compounds having free amino, amido, hydroxy or carboxylic groups can be converted into prodrugs. For instance, free carboxyl groups can be derivatized as amides or alkyl esters. In certain instances, all of these prodrug moieties incorporate groups including but not limited to ether, amine and carboxylic acid functionalities.

Hydroxy prodrugs include esters, such as though not limited to, acyloxyalkyl (e.g. acyloxymethyl, acyloxyethyl) esters, alkoxycarbonyloxyalkyl esters, alkyl esters, aryl esters, phosphate esters, sulfonate esters, sulfate esters and disulfide containing esters; ethers, amides, carbamates, hemisuccinates, dimethylaminoacetates and phosphoryloxymethyloxycarbonyls, as outlined in Advanced Drug Delivery Reviews 1996, 19, 115.

Amine derived prodrugs include, but are not limited to the following groups and combinations of groups:

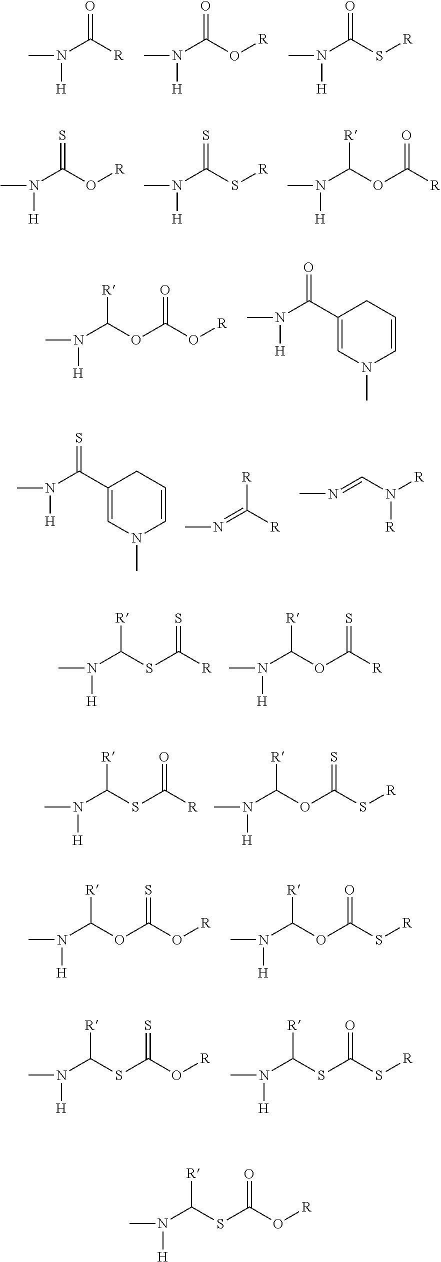

as well as sulfonamides and phosphonamides.

In certain instances, sites on any aromatic ring portions are susceptible to various metabolic reactions, therefore incorporation of appropriate substituents on the aromatic ring structures, can reduce, minimize or eliminate this metabolic pathway.

### Metabolites

In some embodiments, compounds described herein are susceptible to various metabolic reactions. Therefore, in some embodiments, incorporation of appropriate substituents into the structure will reduce, minimize, or eliminate a metabolic pathway. In specific embodiments, the appropriate substituent to decrease or eliminate the susceptibility of an aromatic ring to metabolic reactions is, by way of example only, a halogen, or an alkyl group.

In additional or further embodiments, the compounds (i.e., LPA receptor modulators or antagonists) described herein are metabolized upon administration to an organism in need to produce a metabolite that is then used to produce a desired effect, including a desired therapeutic effect.

### Pharmaceutical Compositions/Formulations

In another aspect, provided herein are pharmaceutical compositions comprising a compound (i.e., LPA receptor modulator or antagonist) as described herein, or a pharmaceutically acceptable salt, polymorph, solvate, prodrug, metabolite, deuteride, N-oxide, stereoisomer, or isomer thereof, and a pharmaceutically acceptable excipient.

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H.A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

Provided herein are pharmaceutical compositions that include a compound described herein (i.e., LPA receptor modulator or antagonist) and at least one pharmaceutically acceptable inactive ingredient. In some embodiments, the compounds described herein are administered as pharmaceutical compositions in which the compound is mixed with other active ingredients, as in combination therapy. In other embodiments, the pharmaceutical compositions include other medicinal or pharmaceutical agents, carriers, adjuvants, preserving, stabilizing, wetting or emulsifying agents, solution promoters, salts for regulating the osmotic pressure, and/or buffers. In yet other embodiments, the pharmaceutical compositions include other therapeutically valuable substances.

A pharmaceutical composition, as used herein, refers to a mixture of a compound (i.e., LPA receptor modulator or antagonist) with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

The pharmaceutical formulations described herein are administered to a subject by appropriate administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

Pharmaceutical compositions including a compound described herein (i.e., LPA receptor modulator or antagonist) are manufactured in a conventional manner, such as, by way of example only, by means of conventional mixing, dissolving, granulating, dragee-making, levigating, emulsifying, encapsulating, entrapping or compression processes.

The pharmaceutical compositions will include at least one compound described herein (i.e., LPA receptor modulator or antagonist) as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, compounds described herein exist in unsolvated form or in solvated forms with pharmaceutically acceptable solvents such as water, ethanol, and the like. The solvated forms of the compounds presented herein are also considered to be disclosed herein.

Pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

Pharmaceutical preparations that are administered orally include push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added.

In certain embodiments, delivery systems for pharmaceutical compounds may be employed, such as, for example, liposomes and emulsions. In certain embodiments, compositions provided herein can also include a mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

Any conventional carrier or excipient may be used in the pharmaceutical compositions of the embodiments. The choice of a particular carrier or excipient, or combinations of carriers or excipients, will depend on the mode of administration being used to treat a particular patient or type of medical condition or disease state.

### Administration of Pharmaceutical Composition

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In some embodiments, compounds described herein (i.e., LPA receptor modulator or antagonists) and compositions thereof are administered in any suitable manner. The manner of administration can be chosen based on, for example, whether local or systemic treatment is desired, and on the area to be treated. For example, the compositions can be administered orally, parenterally (e.g., intravenous, subcutaneous, intraperitoneal, or intramuscular injection), by inhalation, extracorporeally, topically (including transdermally, ophthalmically, vaginally, rectally, intranasally) or the like.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained.

In some embodiments, the pharmaceutical compositions described herein are administered orally.

In other embodiments, pharmaceutical compositions comprising a compound described herein (i.e., lipophilic conjugate of an LPA receptor modulator or antagonist) are administered through subcutaneous injection. In some instances, the lipophilic conjugate of the LPA receptor modulator utilized for administration lacks cellular uptake. In some instances, the lipophilic conjugate of the LPA receptor modulator promotes interaction with the target cell surface and provides an enhanced or sustained effect. In some instances, lipophilic conjugate of the LPA receptor modulator has a long in vivo residence. In some instances, the subcutaneous injection of the lipophilic conjugate of the LPA receptor modulator provides a tissue depot effect.

### Methods

Described herein are certain compounds, compositions, and methods for increasing brown fat differentiation, thereby increasing energy expenditure, insulin sensitization, and/or weight loss. Provided herein in some embodiments are methods of activating or increasing brown fat differentiation, wherein the differentiated brown fat cells increase energy expenditure to treat obesity or an obesity related disorder, e.g., Type II diabetes. Some embodiments provided herein describe methods for increasing energy expenditure in a subject comprising activating or increasing brown fat differentiation in the subject, wherein the differentiated brown fat cells promote energy expenditure thereby increasing energy expenditure in the subject. In some embodiments, the compounds described herein are modulators of BAT development and activity.

In some embodiments is a method of increasing brown fat differentiation in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof. In some embodiments is a method of increasing brown fat differentiation in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator has the structure:

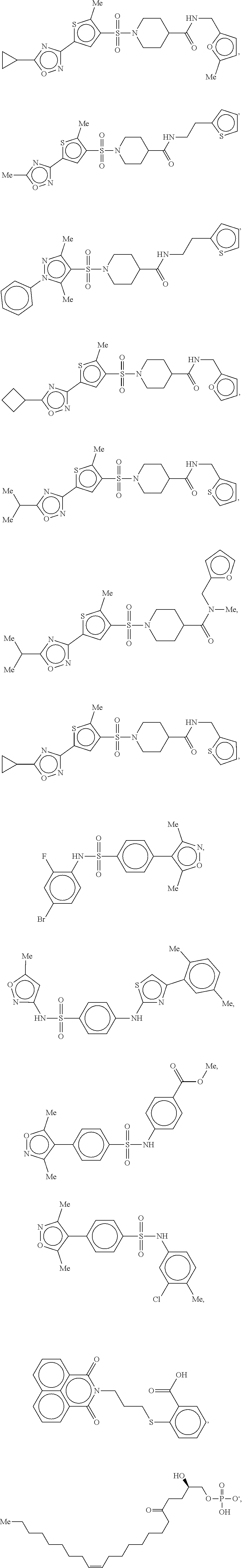

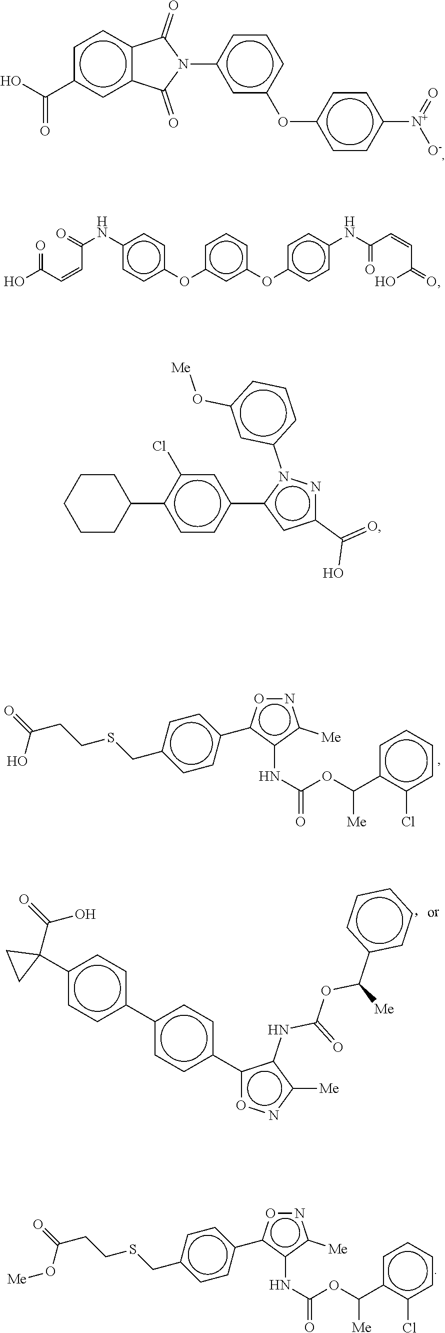

In some embodiments is a method of increasing brown fat differentiation in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator has the structure:

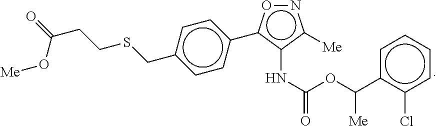

In some embodiments is a method of increasing brown fat differentiation in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator has the structure:

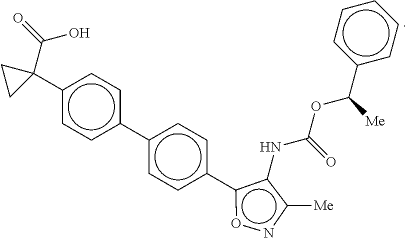

In some embodiments is a method of increasing brown fat differentiation in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator has the structure:

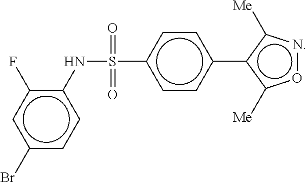

In some embodiments is a method of increasing brown fat differentiation in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator promotes or increases weight loss in the subject. In some embodiments is a method of increasing brown fat differentiation in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator promotes or increases insulin sensitization. In some embodiments is a method of increasing brown fat differentiation in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator is minimally systemically absorbed. In some embodiments is a method of increasing brown fat differentiation in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator is an LPA receptor antagonist.

In some embodiments provided herein is a method for increasing energy expenditure in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, to activate differentiation of the white adipose tissue into brown fat cells in the subject, wherein the differentiated brown fat cells promote energy expenditure thereby increasing energy expenditure in the mammal.

In some embodiments is a method for increasing energy expenditure in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator has the structure:

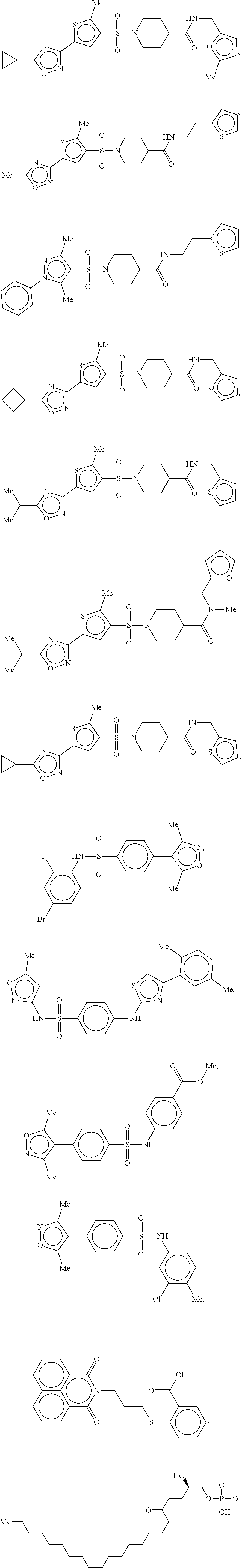

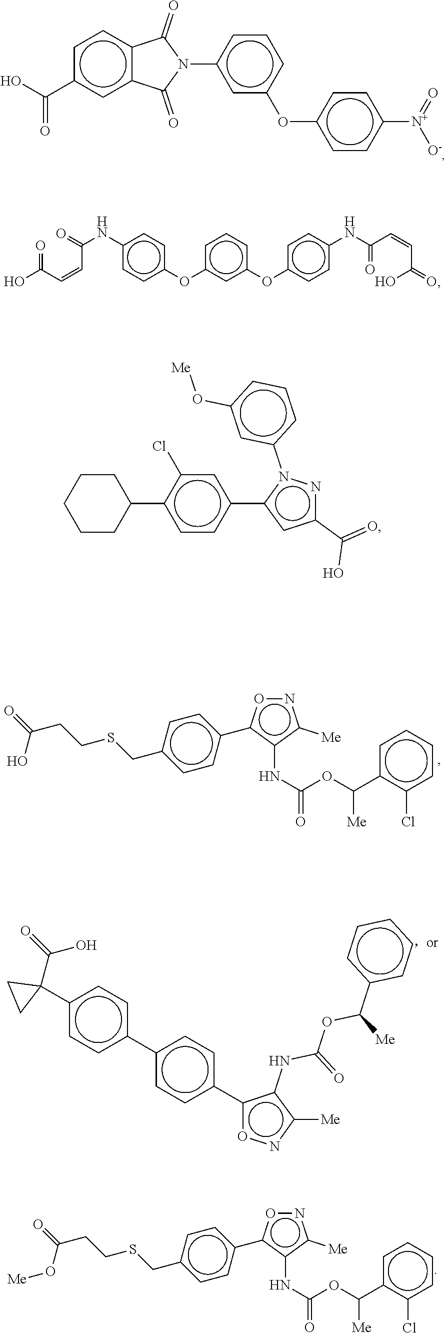

In some embodiments is a method for increasing energy expenditure in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator has the structure:

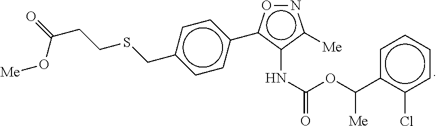

In some embodiments is a method for increasing energy expenditure in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator has the structure:

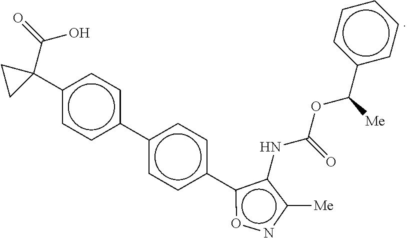

In some embodiments is a method for increasing energy expenditure in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator has the structure:

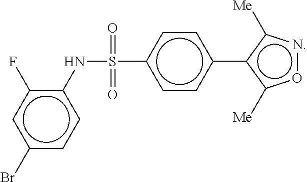

In some embodiments is a method for increasing energy expenditure in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator promotes or increases weight loss in the subject. In some embodiments is a method for increasing energy expenditure in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator promotes or increases insulin sensitization. In some embodiments is a method for increasing energy expenditure in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator is minimally systemically absorbed. In some embodiments is a method for increasing energy expenditure in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, wherein the LPA receptor modulator is an LPA receptor antagonist.

Other embodiments provided herein describe methods for treating obesity or an obesity-related disorder, e.g., Type II diabetes, in a subject comprising administering to the subject an LPA receptor modulator. In some embodiments, the LPA receptor modulator is conjugated to a lipophilic derivative or substituent. In some embodiments, the LPA receptor modulator is conjugated to a lipid or fatty acid derivative. In some embodiments, the LPA receptor modulator is conjugated to a lipid or fatty acid. In some embodiments, the LPA receptor modulator increases respiration and energy expenditure to thereby treat obesity or an obesity-related disorder. In some embodiments, the LPA receptor modulator is an antagonist. In some embodiments, the LPA receptor modulator is an agonist.

Some embodiments provided herein describe methods for providing a metabolic response in a subject comprising administering to the subject an LPA receptor modulator. In some embodiments, the metabolic response is: a) modified expression of a marker selected from the group consisting of: cidea, adiponectin, adipsin, otopetrin, type II deiodinase, cig30, ppar gamma 2, pgc1α, ucp1, elovl3, cAMP, Prdm16, cytochrome C, cox4i1, coxIII, cox5b, cox7a1, cox8b, glut4, atpase b2, cox II, atp5o, ndufb5, ap2, ndufs1, GRP109A, acylCoA-thioesterase 4, EARA1, claudin1, PEPCK, fgf21, acylCoA-thioesterase 3, and dio2; b) modified thermogenesis in adipose cells; c) modified differentiation of adipose cells; d) modified insulin sensitivity of adipose cells; e) modified basal respiration or uncoupled respiration; f) modified hepatosteatosis; g) modified obesity or appetite; h) modified insulin secretion of pancreatic beta cells; i) modified cardiac function; j) modified cardiac hypertrophy; or k) modified muscle hypoplasia. In one embodiment, the compounds described herein (i.e., LPA receptor modulator or antagonist) induces brown fat differentiation.

Other embodiments provided herein describe methods for treating a metabolic disorder in a subject comprising administering to the subject an LPA receptor modulator (e.g., antagonist). In some embodiments, the metabolic disorder is insulin resistance, hyperinsulinemia, hypoinsulinemia, type I diabetes, type II diabetes, protein energy malnutrition, metabolic syndrome, obesity, cardiac disease, early-onset myocardial infarction, osteoarthritis, heart disease, gall bladder disease, gall stones, hypertension, hyperhepatosteatosis, gout, hyperuricemia, fatty liver diseases, non-alcoholic fatty liver disease, polycystic ovarian syndrome, acanthosis nigricans, hyperphagia, endocrine abnormalities, triglyceride storage disease, hyperlipidemia, Bardet-Biedl syndrome, Lawrence-Moon syndrome, Prader-Labhart-Willi syndrome, muscle hypoplasia, neurodegenerative diseases, Alzheimer's disease, or certain types of cancer. In some embodiments, the metabolic disorder is insulin resistance, type I diabetes, type II diabetes, protein energy malnutrition, obesity, or metabolic syndrome. In some embodiments, the metabolic disorder is type I diabetes. In some embodiments, the metabolic disorder is type II diabetes. In some embodiments, the metabolic disorder is protein energy malnutrition. In some embodiments, the metabolic disorder is obesity. In other embodiments, the metabolic disorder is hyperlipidemia. In some embodiments, the compounds and compositions described herein are used to increase insulin sensitization in subjects in need thereof.

Also envisioned is the treatment of patients who desire treatment for aesthetic reasons (i.e. to maintain a desired weight, BMI, or appearance) even if they are at a healthy weight or BMI prior to treatment. In some embodiments, the compounds and compositions described herein are used for weight loss or weight maintenance. In some embodiments, the compounds and compositions described herein are used to protect a subject against the development of obesity. In some embodiments, the therapeutics described herein are used to protect a subject from diet-induced obesity.

Other embodiments provided herein describe a method of inducing or promoting brown fat differentiation in a subject, the method comprising administering to the subject an LPA receptor modulator (e.g., antagonist) conjugated to a lipophilic derivative or substituent as described herein, or a pharmaceutically acceptable salt, solvate, polymorph, prodrug, metabolite, deuteride, N-oxide, stereoisomer, or isomer thereof. In some embodiments, the compounds and compositions described herein demonstrate improved potency and/or PK properties and are useful for increasing brown fat differentiation in a subject in need thereof.

In some embodiments, the compounds described herein are not systemically absorbed. In some embodiments, the compounds described herein are minimally systemically absorbed. In some instances, side effects due to systemic absorption are reduced, minimal, or absent.

### EXAMPLES

### Brown Cell Differentiation

Media and Reagents

OF Media included 1% Anti-Anti, 3.3 mM biotin, 1.7 mM panthotenate, and DMEM:F12 (1:1) media. Growth media included OF Media and 10% Fetal Bovine Serum. Differentiation induction media included OF Media, 10 μg/mL Insulin, 0.5 mM IBMX, and 1 μM Dexamethasone. The maintenance media included OF media and 10 μg/mL Insulin.

### Cell Culture

Human Simpson-Golabi-Behmel syndrome (SGBS) preadipocyteswere cultured in T225 flasks containing Growth Media. Upon passaging, cells were trypsinized, collected, spun down at 1250 g for 5 minutes at 20° C. to pellet. Cells were then resuspended in growth media before seeding cells at optimal density to maintain 75-80% confluency before passaging every 3-4 days.

### Induction of Differentiation

Compounds at varying concentrations (starting at 20 uM with 1:3 dilution) were spotted onto 384-well black clear bottom plates (Greiner). The negative control consisted of DMSO while positive controls contained 2 uM of Rosiglitazone (Cayman). 50 μL of SGBS cells in induction were seeded directly on top of compound at a concentration of 1.1×10⁵ cells/mL (˜5500 cells/well). After 4 days of induction, media was changed to 50 μL of maintenance media. Every 2-4 days after, media was changed again with 50 μL maintenance media until day 11.

Staining

After 11 days of differentiation, cells were fixed with 10 μL of 32% Paraformaldehyde for 10 minutes, followed by 10 μL of 7% Triton X-100 for another 10 minutes. Media was then aspirated down to 5 μL, 25 μL of Blocking Buffer 1 (2% BSA and 2.5% goat serum) was added, and plates were incubated for 1 hour. Plates were then aspirated down to 5 μL and 25 μL of primary antibody was added for 16 hour incubation. Primary antibody containing UCP1 (Sigma; Rabbit; 1:100) was diluted in Blocking Buffer 2 (2% BSA).

The next day, plates were washed 3 times with 1× PBS and 25 μL of secondary antibody were added. Secondary antibody containing Hoechst (1:6000), Alex Fluor 647 goat Anti-Rabbit (1:1000), and HCS LipidTOX Green Neutral Lipid Stain (Life Technologies; 1:800) were diluted in Blocking Buffer 2 (2% BSA). After 1 hour of incubation at room temperature, plates were washed 3 times with 1× PBS and left in 50 μL of PBS. Plates were then sealed and imaged.

Imaging

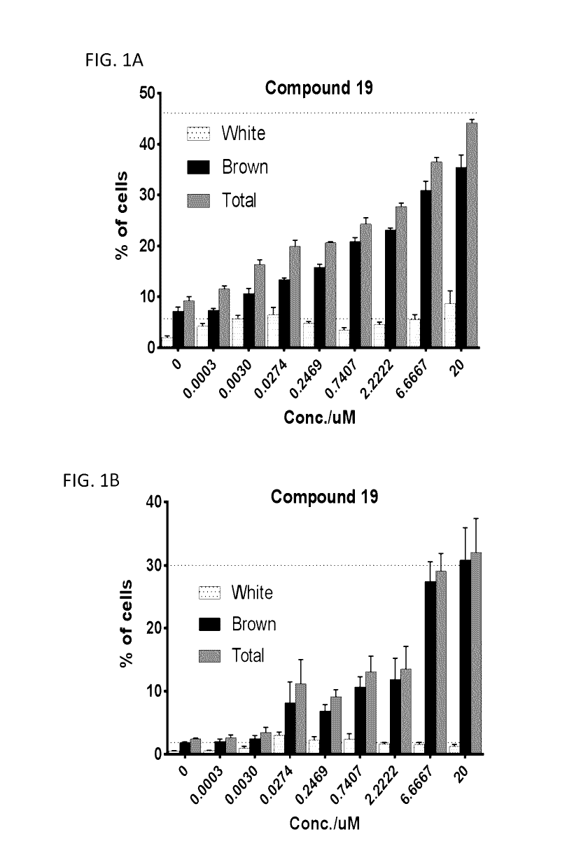

Plates were imaged using CellInsight CX5 High Content Screening (HCS) Platform. Total cell number was detected using Hoechstand images were used to quantify total adipocytes (met defined threshold of lipidtox staining intensity). Of the total adipocytes, those that met a defined threshold of UCP1 staining intensity were labeled ‘brown’ and those that failed to meet the defined threshold of UCP1 staining intensity were labeled ‘white’. Data are shown in Table 1 and FIG. 1A.

TABLE 1

| | % Total | | | % White | | | % Brown | | | UCP1 fold | | |
|---|---|---|---|---|---|---|---|---|---|---|---|---|
| Compound 8 | 32% | 42% | 48% | 6% | 9% | 3% | 26% | 33% | 45% | 2.9x | 2.6x | 4.7x |
| Compound 2 | 37% | 41% | 41% | 9% | 8% | 7% | 29% | 34% | 34% | 2.2x | 2.8x | 2.9x |
| Compound 3 | 30% | 36% | 36% | 10% | 8% | 5% | 20% | 28% | 30% | 1.6x | 2.1x | 2.9x |
| Compound 1 | 21% | 29% | 34% | 6% | 3% | 4% | 15% | 27% | 30% | 1.2x | 2.6x | 2.7x |
| Compound 9 | 23% | 27% | 37% | 5% | 6% | 6% | 18% | 21% | 31% | 1.5x | 1.9x | 2.7x |
| Compound 4 | 22% | 30% | 35% | 5% | 7% | 5% | 17% | 22% | 30% | 1.5x | 1.6x | 2.6x |
| Compound 5 | 21% | 29% | 36% | 3% | 7% | 7% | 18% | 22% | 28% | 1.8x | 1.8x | 2.2x |
| Compound 10 | 23% | 29% | 38% | 6% | 8% | 10% | 17% | 21% | 29% | 1.5x | 1.5x | 2.0x |
| Compound 6 | 20% | 29% | 34% | 6% | 6% | 5% | 14% | 23% | 29% | 1.2x | 1.7x | 2.4x |
| Compound 7 | 19% | 27% | 33% | 4% | 5% | 4% | 15% | 21% | 28% | 1.4x | 1.8x | 2.4x |
| Compound 11 | 23% | 28% | 36% | 4% | 6% | 11% | 18% | 22% | 24% | 1.7x | 1.8x | 1.7x |

Similar experiments were performed as outlined above except MitoTracker staining was performed instead of UCP1. 25 nM of MitoTracker was added onto live cells and incubated at 37° C. for 30 minutes. After staining, cells were washed 2 times with fresh media then fixed with 5% paraformaldehyde for 10 minutes at RT. Lipid staining and imaging were performed as above. Images were used to quantify total adipocytes (met defined threshold of lipidtox staining intensity). Of the total adipocytes, those that met a defined threshold of mitotracker staining intensity were labeled ‘brown’ and those that failed to meet the defined threshold of mitotracker staining intensity were labeled ‘white’. Data are shown in FIG. 1B.

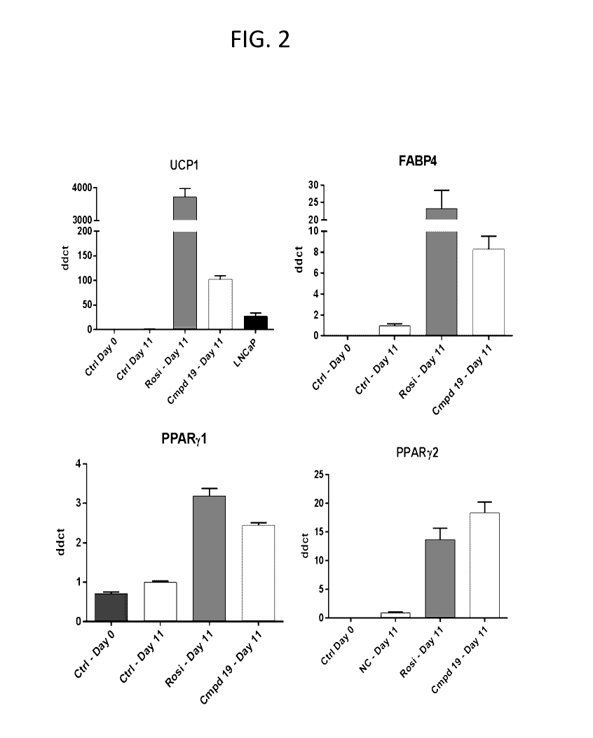

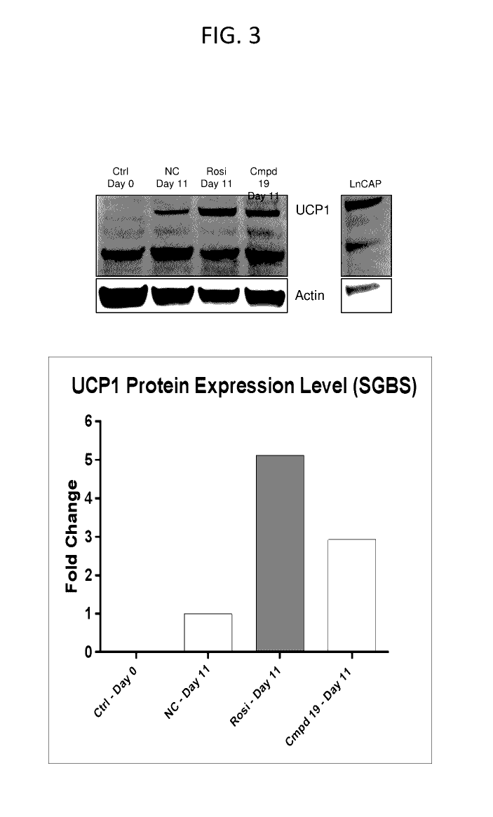

SGBS cells plated in 6-well plate at 450,000 cells per well were differentiated as described above but with a single concentration of test compound (10 μM). RNA was isolated using Qiagen RNeasy mini kit and manufacturer's protocol was followed. The concentration of eluted RNA was measured using Nanodrop reading. 20 ng of RNA was reversely transcribed into cDNA using Quanta qScript SXLT cDNA supermix. qPCR was done in triplicates on the ViiA 7 real-time PCR system (Thermo Fisher) using SYBR advantage qPCR premix (Clontech). Oligonucleotides for human gene expression studies were as follows: FABP4, 5′-TGCAGCTTCCTTCTCACCTT-3′ (SEQ ID NO: 1) (sense) and 5′-GGCAAAGCCCACTCCTACTT-3′ (SEQ ID NO: 2) (antisense); HPRT, 5′TGACACTGGCAAAACAATGCA-3′ (SEQ ID NO: 3) (sense) and 5′-GGTCCTTTTCACCAGCAAGCT-3′ (SEQ ID NO: 4) (antisense); PPARγ1, 5′-CGTGGCCGCAGATTTGA-3′ (SEQ ID NO: 5) (sense) and 5′AGTGGGAGTCTTCCATTAC-3′ (SEQ ID NO: 6) (antisense); PPARγ2, 5′-GAAAGCGATTCCTTCACTGAT-3′ (SEQ ID NO: 7) (sense) and 5′-TCAAAGGAGTGGGAGTGGTC-3′ (SEQ ID NO: 8) (antisense); and 5′UCP1, 5′-CCAACTGTGCAATGAAAGTGT-3′ (SEQ ID NO: 9) (sense) and 5′-CAAGTCGCAAGAAGGAAGGT (SEQ ID NO: 10). Data are shown in FIG. 2. In parallel experiments, cells were instead lysed using RIPA Buffer (Thermo Fisher) supplemented with protease inhibitor cocktail set I (EMD Millipore). Protein samples were separated on 4-20% SDS-PAGE (Thermo Fisher) and transferred for 90 min at 40 V onto PVDF membranes (Millipore). Membranes were blocked using Odyssey blocking buffer (Licor). Membranes were incubated with primary antibodies at 4° C. overnight then washed with PBS-Tween and incubated with secondary antibodies for 1 h at room temperature. Western blots were imaged and quantitated using the Odyssey infrared imaging system. Antibodies: UCP1 (Abcam ab155117, 1:500), FABP4 (Abcam ab66682, 1:1000), and β-actin (Santa Cruz sc-47778, 1:1000). Data are shown in FIG. 3.

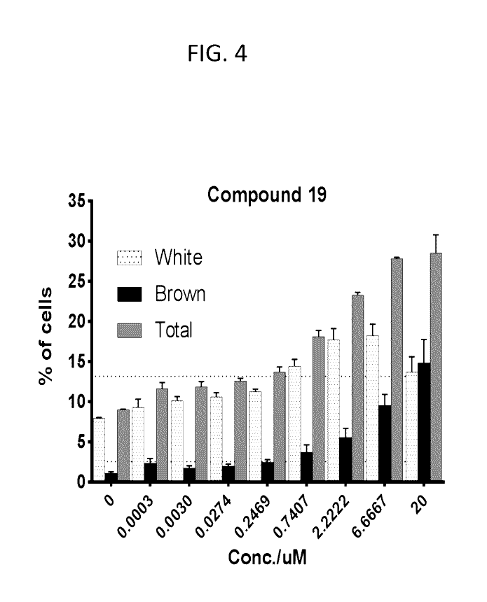

Similar experiments in 384-well format as outlined above for SGBS cells were repeated with human primary preadipocytes plated at a concentration of 8×10⁴ cells/mL. Upon reaching 100% confluency, cells were treated with compound and human Induction Medium (Preadipocyte Medium with 10 μg/mL Insulin, 0.5 mM IBMX, and 1 μM Dexamethasone). After 4 days with induction medium, media was removed and replaced with maintenance medium (Preadipocyte Medium with 10 μg/mL Insulin) until day 11. Staining procedures were as above. Data are shown in FIG. 4.

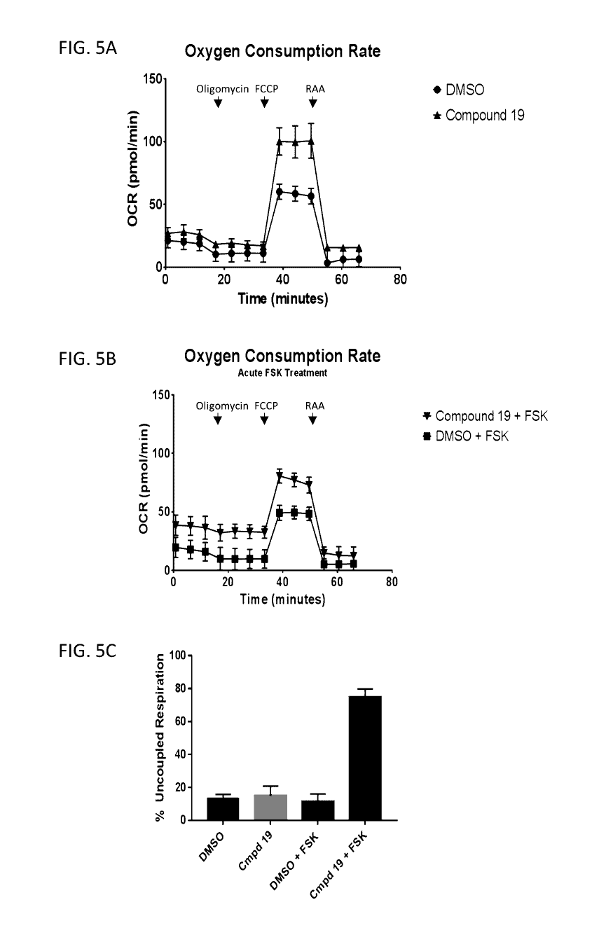

SGBS preadipocytes were plated at a concentration of 1×10⁵ cells/ml in 1% gelatin-coated XF 96-well cell culture microplates (Seahorse Bioscience). At confluence, cells were treated with compound and differentiated into adipocytes as described above. Day 11 adipocytes were subjected to Seahorse experiments. Cells were incubated with XF base medium containing 2 mM Glutamine, 1 mM sodium pyruvate, and 18 mM glucose for 1 hour. The oxygen consumption rate (OCR) from adipocytes were measured using the Seahorse flux analyzer XF96 with sequential injections of 2 μM oligomycin, 1 μM FCCP, and 0.5 μM RAA. Data are shown in FIG. 5A. In parallel experiments, cells were incubated in base medium that also included 10 μM forskolin before measuring OCR rates. Data are shown in FIG. 5B. Total basal mitochondrial respiration is measured as OCR prior to Oligomycin injection. ATP coupled respiration is calculated based on the decrease in OCR after oligomycin injection. Maximal respiration is measured after addition of FCCP. Percent uncoupled respiration is the portion of total basal mitochondrial respiration that was not sensitive to Oligomycin injection. Data are shown in FIG. 5C.

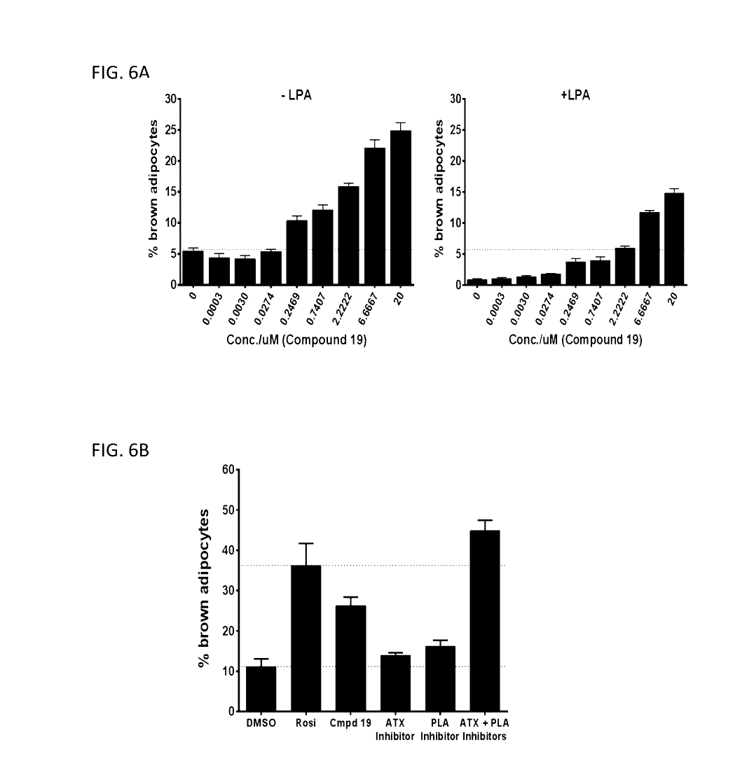

Compound 19, Rosiglitazone, ATX inhibitor HA155 (Sigma-Aldrich), PLA inhibitor PACOCF3 (Tocris), or both HA155 and PACOCF3 were pre-spotted in replicates onto 384-wp and SGBS preadipocytes were plated on top and differentiated out to day 11 and stained for UCP1 as described above. Final concentration of Rosi was 2 uM and all other compounds 10 μM. Data are shown in FIG. 6B.

SGBS preadipocytes were plated in 384 plates as previously described but using induction media with or without the addition of 10 μM lysophosphatidic acid (LPA) for the first 2 days. On day 3, cells were treated with 10 μL of 6× concentration of compound 19 (final concentration was a 1:3 serial dilution starting at 20 μM) again with or without the addition of LPA. On day 5, media was changed to maintenance media until day 11. Staining and analysis of brown adipocytes based on UCP1 and lipid performed as described previously. Data are shown in FIG. 6A.

1. A method of increasing brown fat differentiation in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof.

2. The method of claim 1, wherein the LPA receptor modulator has the structure:

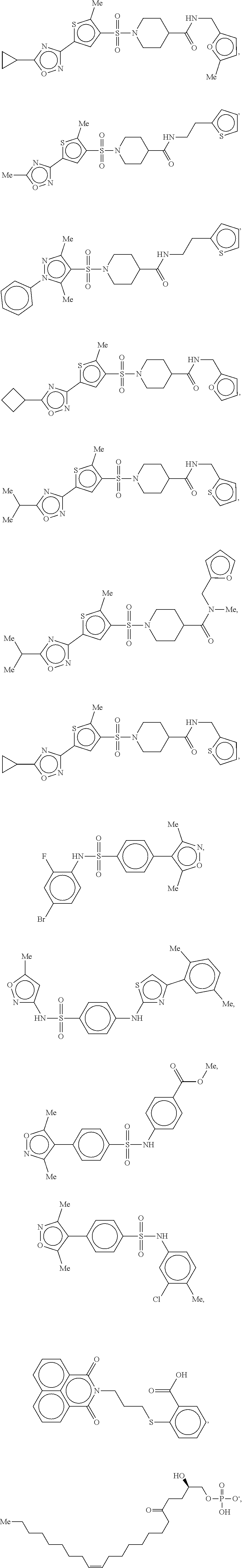

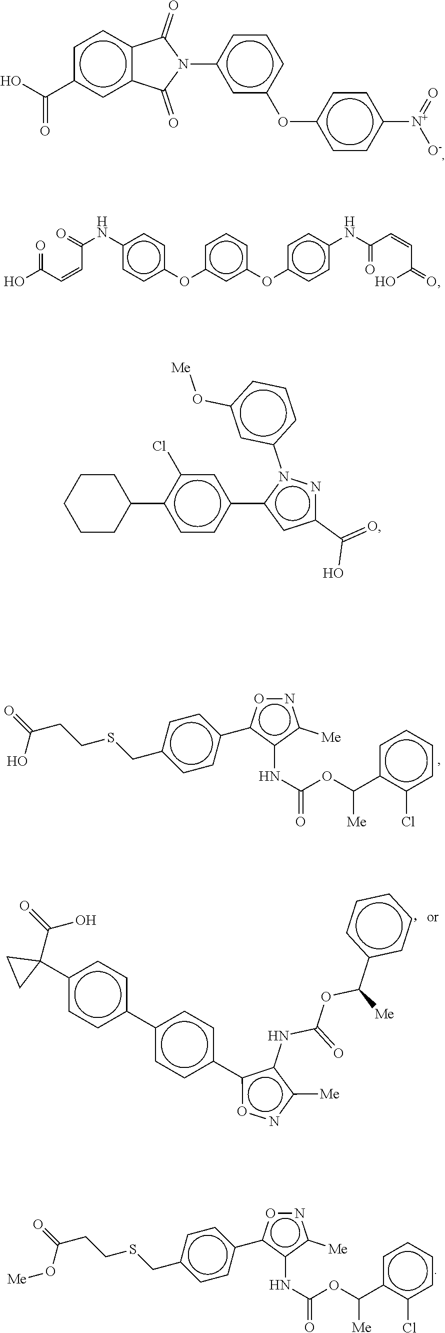

3. The method of claim 1, wherein the LPA receptor modulator has the structure:

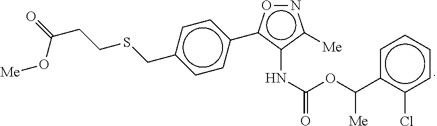

4. The method of claim 1, wherein the LPA receptor modulator has the structure:

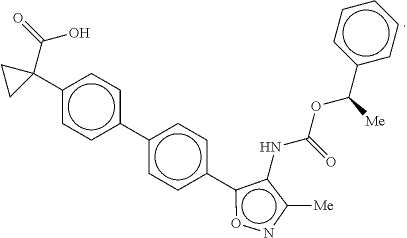

5. The method of claim 1, wherein the LPA receptor modulator has the structure:

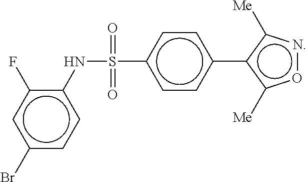

6. The method of claim 1, wherein the LPA receptor modulator promotes or increases weight loss in the subject.

7. The method of claim 1, wherein the LPA receptor modulator promotes or increases insulin sensitization.

8. The method of claim 1, wherein the LPA receptor modulator is minimally systemically absorbed.

9. The method of claim 1, wherein the LPA receptor modulator is an LPA receptor antagonist.

10. A method for increasing energy expenditure in a subject in need thereof, the method comprising administering to the subject an LPA receptor modulator, or a pharmaceutically acceptable salt, solvate, polymorph, prodrug, metabolite, deuteride, N-oxide, stereoisomer, or isomer thereof, to activate differentiation of the white adipose tissue into brown fat cells in the subject, wherein the differentiated brown fat cells promote energy expenditure thereby increasing energy expenditure in the mammal.

11. The method of claim 10, wherein the LPA receptor modulator has the structure:

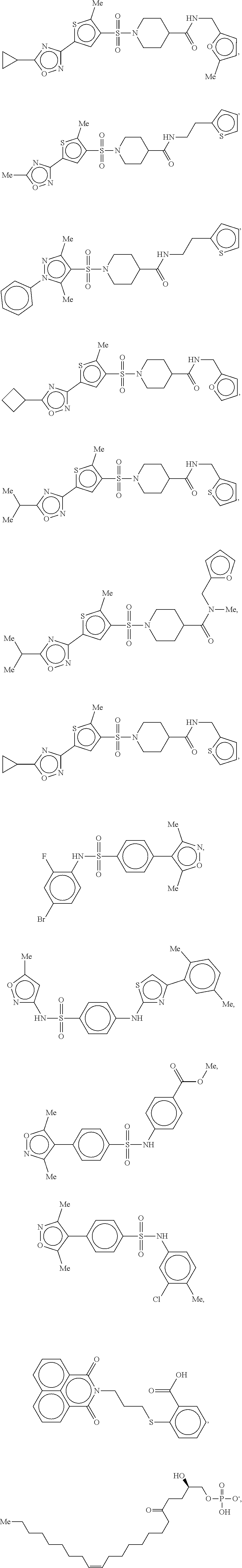

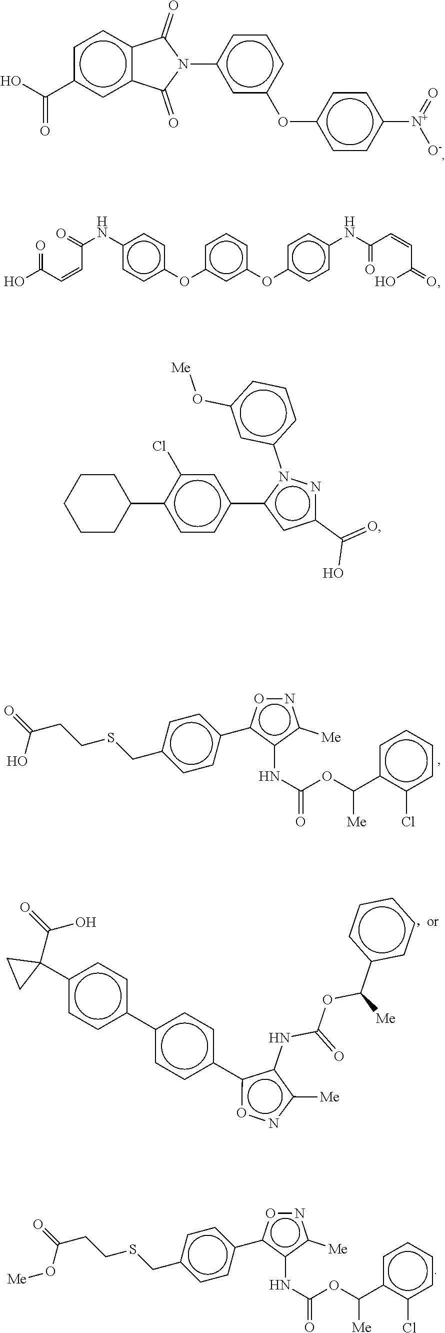

12. The method of claim 1, wherein the LPA receptor modulator has the structure:

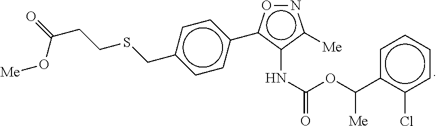

13. The method of claim 10, wherein the LPA receptor modulator has the structure:

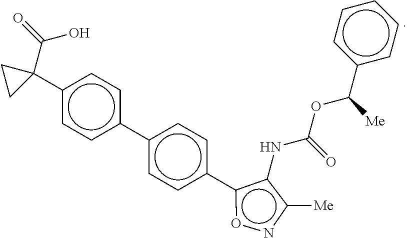

14. The method of claim 10, wherein the LPA receptor modulator has the structure:

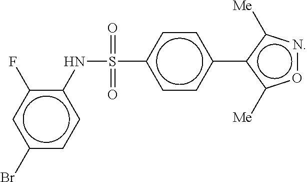

15. The method of claim 10, wherein the LPA receptor modulator promotes or increases weight loss in the subject.

16. The method of claim 10, wherein the LPA receptor modulator promotes or increases insulin sensitization.

17. The method of claim 10, wherein the LPA receptor modulator is minimally systemically absorbed.

18. The method of claim 10, wherein the LPA receptor modulator is an LPA receptor antagonist.


SEQUENCE LISTING (10 sequences)

SEQ ID NO: 1 - DNA, 20 bp, Homo sapiens
tgcagcttccttctcacctt

SEQ ID NO: 2 - DNA, 20 bp, Homo sapiens
ggcaaagcccactcctactt

SEQ ID NO: 3 - DNA, 21 bp, Homo sapiens
tgacactggcaaaacaatgca

SEQ ID NO: 4 - DNA, 21 bp, Homo sapiens
ggtccttttcaccagcaagct

SEQ ID NO: 5 - DNA, 17 bp, Homo sapiens
cgtggccgcagatttga

SEQ ID NO: 6 - DNA, 19 bp, Homo sapiens
agtgggagtcttccattac

SEQ ID NO: 7 - DNA, 21 bp, Homo sapiens
gaaagcgattccttcactgat

SEQ ID NO: 8 - DNA, 20 bp, Homo sapiens
tcaaaggagtgggagtggtc

SEQ ID NO: 9 - DNA, 21 bp, Homo sapiens
ccaactgtgcaatgaaagtgt

SEQ ID NO: 10 - DNA, 20 bp, Homo sapiens
caagtcgcaagaaggaaggt